(19) United States
(12) Patent Application Publication
(10) Pub. No.: US 2017/0056751 A1
(43) Pub. Date: Mar. 2, 2017

(54) HEADGEAR WITH EAR PROTECTION AND HAIR CONTAINMENT

(71) Applicant: Danmar Products, Inc., Ann Arbor, MI (US)

(72) Inventor: John L. Marchello, Ann Arbor, MI (US)

(21) Appl. No.: 14/834,972

(22) Filed: Aug. 25, 2015

(51) Int. Cl. A63B 71/10; A61F 11/06; A42C 5/04; A42B 1/08

(52) U.S. Cl. CPC A63B 71/10; A42B 1/08; A61F 11/06; A42C 5/04

(57) ABSTRACT

An improved head gear with ear guard inserts integrated into a hair covering cap, particularly for amateur wrestlers, comprising a single, jaw strap, attachment and two ear guard inserts that have unique features and enhance comfort and natural hearing when held in a position appropriate for use by the cap material and methods of construction.

### FIELD OF THE INVENTION

This invention relates generally to protective headgear and, in particular, to a head worn article including an ear guard assembly that fulfills hearing and ventilation functions.

### BACKGROUND OF THE INVENTION

The outer ears of wrestlers and athletes involved in other contact sports are subjected to contact of a nature that can cause hemorrhaging and permanent damage (Giffin). As a primary means of protecting against such injury, competitors commonly wear head gear that holds ear guards in place.

Ear guard positioning methods should require low effort with little or no impact to other ear guard functions and the act of wrestling. Furthermore, head gear must remain attached for a contest to continue.

Headgear designs that employ a plurality of individual straps, each attached to ear guards have a long and widespread presence in prior art (Johnson) (Purnell). These ear guards provide a protective enclosed space positioned over each outer ear, and incorporate a method of positioning and limiting slippage of each ear guard. The designs protect the ears from impact, have comfort cushioning, provide air paths for hearing and ventilation, are cleanable, and limit the impact of the headgear on the activity of grappling to varying degrees.

A less developed area of development involves the employment of a one piece (e.g., knit or elastomeric) elastic web (Reese) (Dobbs and Empero). In these examples, appropriately flexible one-piece elastic web type of headgear addresses difficulties of adjustment.

An additional role of headgear is to limit the effect of athlete's hair on competition—the hair of wrestlers must meet a grooming standard (e.g., length and abrasive quality) or a legal hair cover must be worn (National Federation of State High School Associations). This has typically been accomplished through use of a separate covering (e.g., The “Slicker” (Cliff Keen), “Hair Cap” (Matman) or Hair Cover (Brute, Adidas)). To address any difficulties with maintaining the position of these hair covers during contests, hair covers have recently become required by rule (National Federation of State High School Associations) to be attached in some manner to the headgear to maintain their intended position during matches.

To meet requirements as a hair cover, equipment must provide continuous coverage of the hair and hold it in a position that complies with the grooming standard. Headgear with gapless coverage that conceals the hair of the wearer is not very common in prior art (Ihnat) (Dobbs and Empero). Ihnat presents an invention constructed of an elastic material that eliminates fasteners altogether and relies on a seamless elastic cover to adjust and secure the headgear at the chin. The elastic cover has rigid (or semi-rigid) plate/pad assemblies held securely in pockets over the user's ears. The Dobbs, et al. headgear uses a hook and loop fastener closure for adjustment and attachment and a foam ring surrounding the ear of the wearer.

Individual contestants may prefer different levels or qualities of sound while participating in events and it has been stated that some designs in the prior art muffle, attenuate, or reverberate the sound in an objectionable manner. Ear guard assemblies in the prior art address sound include apertures of various shapes, sizes, quantities, and arrangements on the outer portion of the ear guard that result in varied effects on perception of sound level and quality.

It is common practice for sports teams and individuals to bolster support through the application of graphic design (e.g., colors, logos, slogans) on sports equipment. However, the nature in which the decoration can be executed can be limited by the methods chosen to fulfill the hearing, ventilating and material durability functions of the ear guard assembly.

It is recognized that keeping wrestling gear clean may be the most effective means of preventing outbreaks of skin infections (William L. Dienst, Dightman and Dworkin). Therefore it is desireable to minimize effort required to keep the headgear clean.

### SUMMARY OF THE INVENTION

In broad and general terms, this invention provides an ear guard assembly that fulfills hearing and ventilation functions. The invention reduces adjustment effort to a single buckle at the jaw strap, maintains or improves—hearing and ventilation functions by positioning semi-rigid ear protection elements within the pockets of a breathable, stretchable cap that completely covers a contestant's hair. Encapsulation of the ear guard inserts in the material of the cap makes wearing the headgear more comfortable and protects competitors from contact with headgear straps or hard edges. The construction of the head gear cover enables the use of custom materials and graphics to foster individual and team spirit. The headgear may be disassembled more easily for laundering of the cap and washing of the insert or alternatively may be worn into the shower and cleaned when showering to reduce the build-up of soil and/or infectious biological matter.

### BRIEF DESCRIPTION OF DRAWINGS

FIG. 1 is a perspective view of a headgear 1 (as worn), illustrating the cap 2 and orientation of the inserts 3 within the cap;

FIG. 2 is a view illustrating the tension-transfer 8 from the jaw strap 4 through the bands 7 and 27 to the panels 30-32 to the insert pocket perimeter stitching 5;

FIG. 3 is a view showing the interior of the cap 2 and insertion of inserts 3;

FIG. 4 is a view looking out from the wearers head at one of the inserts 3;

FIG. 5 is a cross-sectional view of one insert 3 taken along line 5-5 of FIG. 4;

FIG. 6 is a side view of a headgear 1 illustrating the airflow through cap 2 and around inserts 3;

FIG. 7 is a partial view illustrating the jaw strap 4 of the head gear 1 shown in FIGS. 1, 2, 3, and 6; and

FIG. 8 is a cross-sectional view of one insert 3 taken along line 8-8 of FIG. 6.

### DETAILED DESCRIPTION OF THE INVENTION

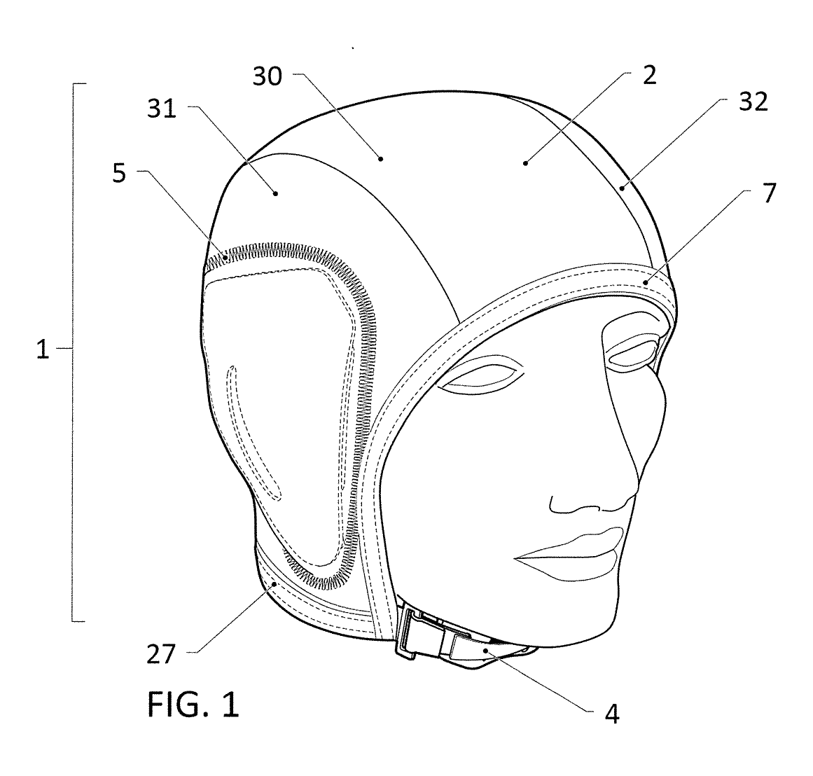

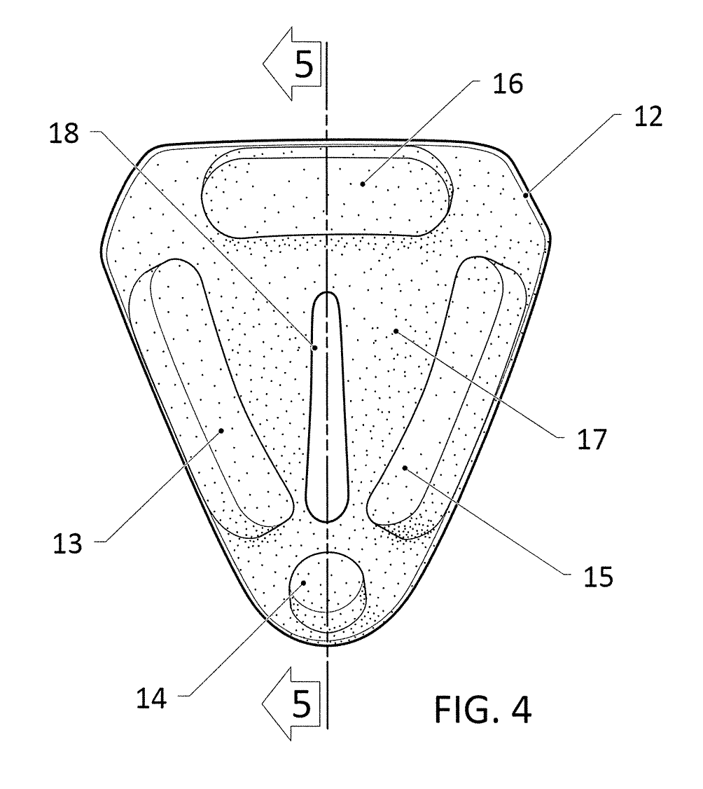

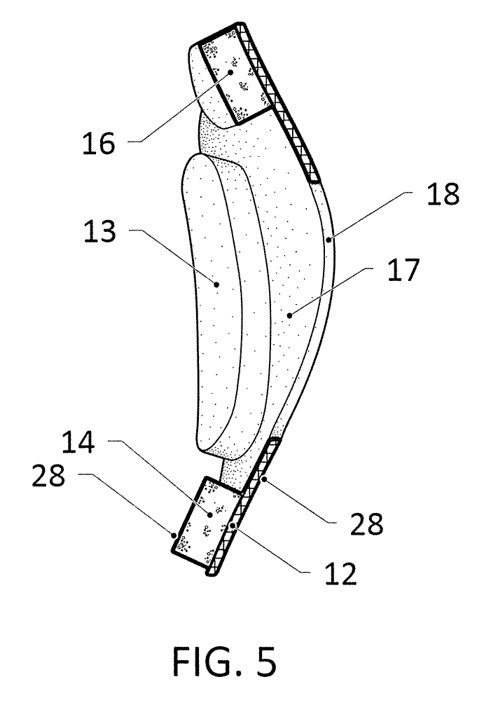

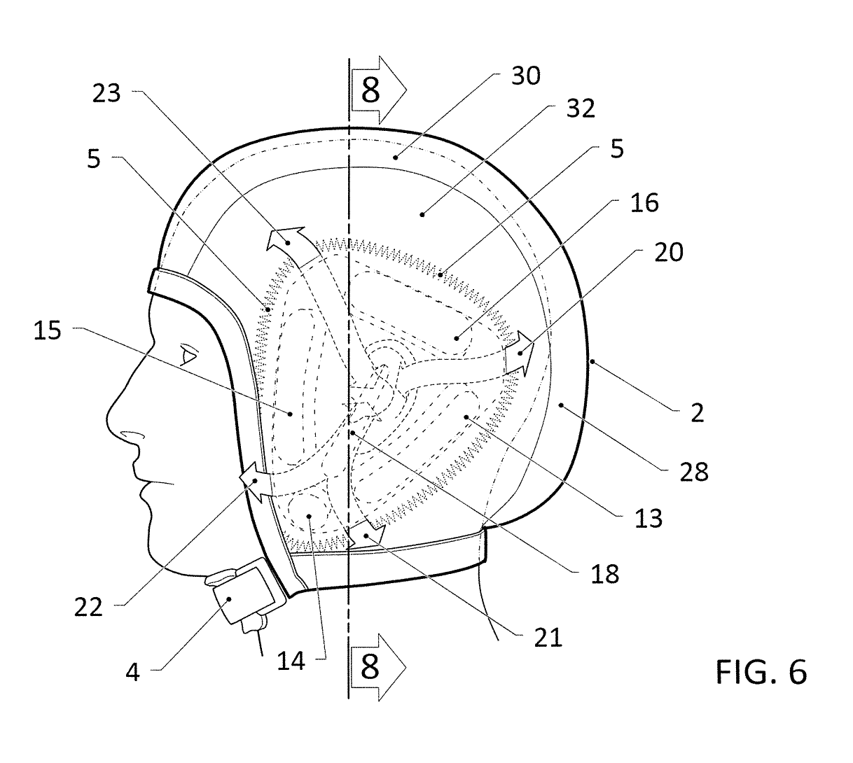

One embodiment of the invention incorporated into an improved head gear assembly 1 particularly adapted for a wrestling ear guard is shown pictorially in FIGS. 1 through 8. Head gear 1 comprises a cap 2 and two (2) ear guards 3. The cap 2 may be formed from any of various materials that provide the elastic and breathability characteristics necessary to hold the inserts 3 in place. A preferred embodiment includes an arrangement of a center panel 30 and side panels 31 and 32. First, this construction method uses highly breathable material provides comfortable, porous layer between the non-porous ear guards 3 and the user's ears and allows for the free exchange of air and water vapor through the panels 30, 31, and 32. Secondly, the panels simultaneously offer enough stretch to retain long hair and passive adjustment yet enough resistance to maintain the position of the ear guards 3 on the user's head. In addition, the fabric used to construct the covering does not irritate the skin of fellow competitors.

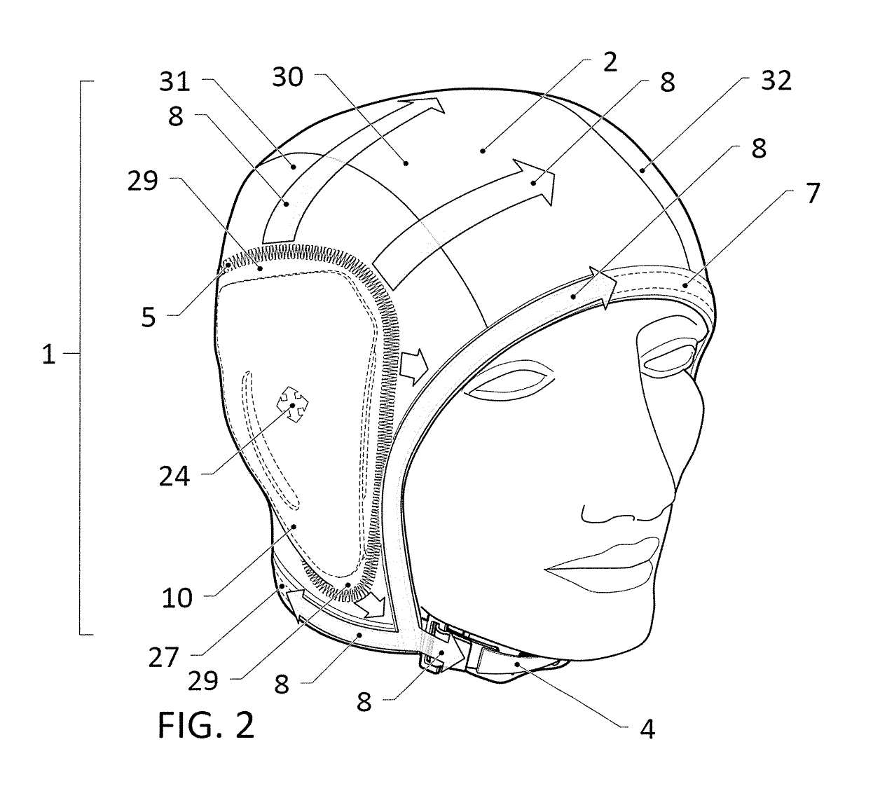

Cap 2 includes pockets 6 that are sized to provide only enough positional freedom 24 for the cap 2 and insert 3 to position the insert 3 as is appropriate when jaw strap 4 is adjusted for use by the wearer. The perimeter of the flaps 25 and 26 that create the pocket 6 are attached to the main body of the cap with a stitch 5 that allows the necessary flexibility for cap performance without overstressing the material. The jaw strap 4 adjustment limits the amount that the cap 2 material must stretch to be functional—reducing the requirements placed on the material and increasing freedom for fabric selection. The face band 7 and neck band 27 are formed in such a manner as to be of greater stiffness than the outer surface of the cap 2 so as to more effectively transmit attachment loads and retain the user's hair in space 28. The insert 3 “floats” 24 (FIG. 2) within the pocket 6 due to space 29 to accommodate the location of the wearer's ear while being held against the head by the outer layer 10, face opening band 7, and neck band 27 of the cap 2 and jaw strap 4 due to the set of tensile forces 8.

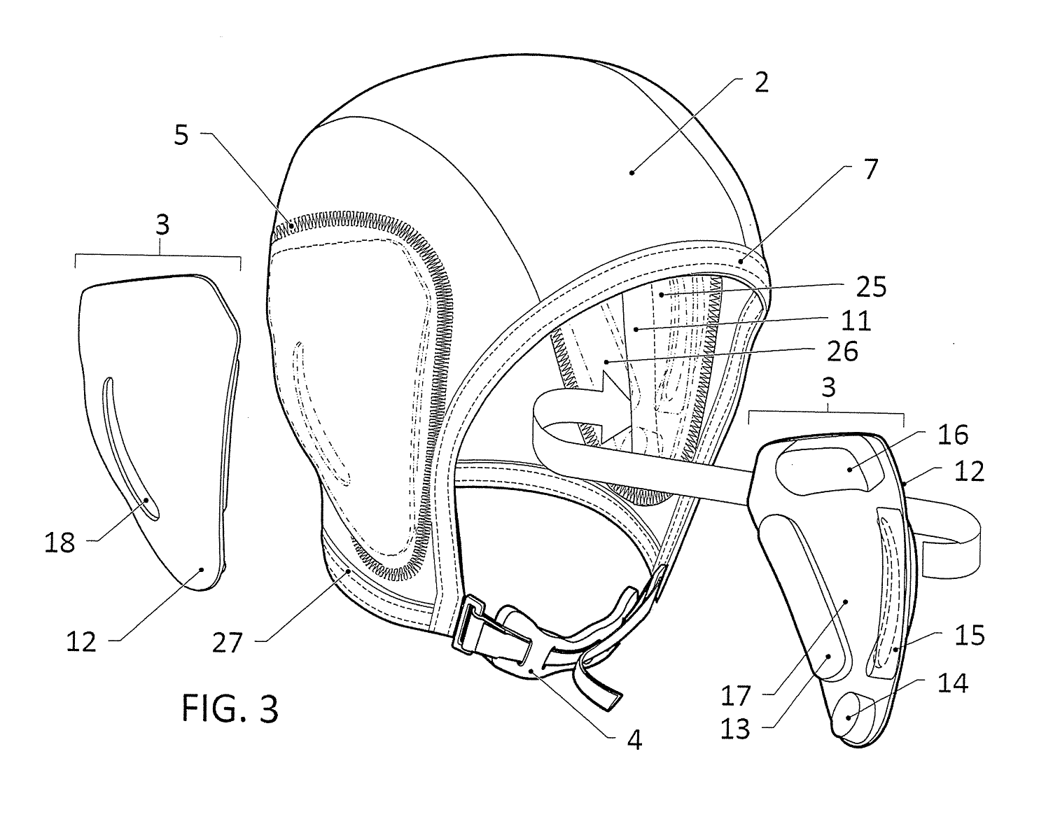

Best seen in FIG. 3, the direction of the overlap 11 in insert pocket 6 is preferentially in the same direction as the primary line of force used to attach headgear 1 to head with jaw strap 4 to resist opening when stressed during use. The flaps of overlap 11 may or may not be secured with a fastener that is appropriate for use next to the wearer's ear.

Alternatively, portions of the cap 2 may be omitted to allow added ventilation (if hair covering is not required) and still represent this invention.

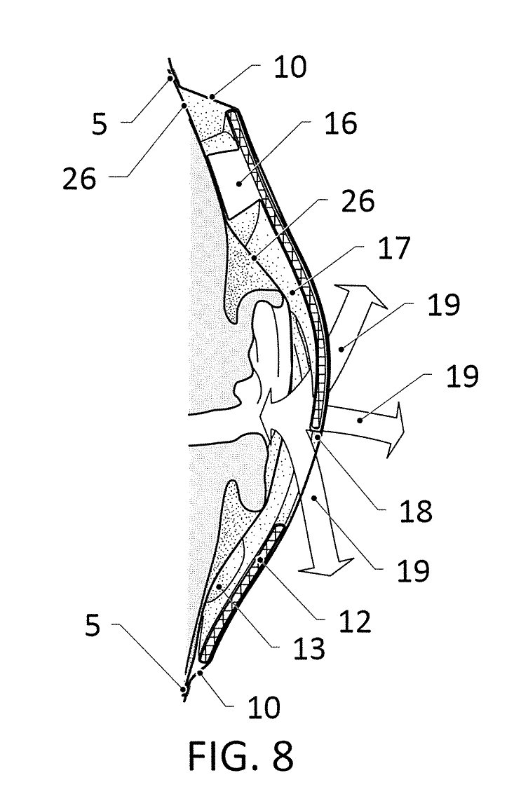

Each insert 3 comprises a rigid or semi-rigid structural reinforcement 12, spacer pads 13-16 as best illustrated in FIGS. 3, 4 and 5. The inner surface 17 of the structural reinforcement 12 is concave in shape so as to provide space for the ear of the wearer and has preferentially elongated slot 18 to provide an air path 19 (FIG. 8) to convey sound to the ear from a multitude of directions and ventilate the wearer's head. Inserts 3 are supported against the head at discrete skeletal areas surrounding the elongated area of the ear rather than in a continuous annular area as in previous art. The perimeter of structural reinforcement 12 is shaped to support a multitude of resilient pads 13-16 at locations appropriately positioned to nest over the wearer's cranium, cheekbones, and jaw to cushion loads from the outside (e.g., during wrestling contests).

In addition to providing purchase at areas of the wearer's skull and jaw, pads 13-16 create additional paths 20-23 for air to circulate through the insert from a multitude of directions rather than only in and out of the insert along the direction of the wearer's ear canal, providing additional airflow from that found provided in prior art. This modification improves comfort by offering greater opportunity for evaporation of perspiration and allows sound to flow through the headgear from a multitude of directions to improve quality of hearing.

In a preferred embodiment, the inserts 3 are subsequently coated with vinyl 28 in a manner sufficient to smooth any abrupt edges on the structural plate 12 that may have resulted from manufacturing or handling and protect the pads 13-16 from absorbing substances that could encourage growth of biologicals.

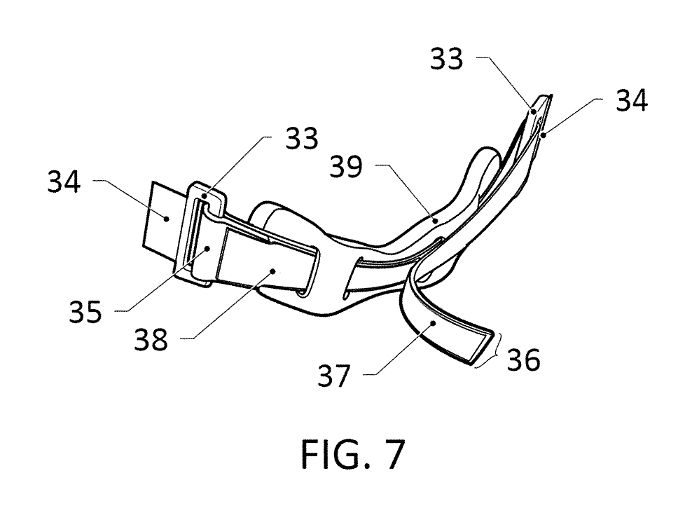

The preferred arrangement of components for the jaw strap 4 are shown in FIG. 7. The D-rings 33 are sewn into the cap 2 through flaps 34. Strap 35 is preferably constructed of nylon webbing and attached to d-ring 33 by any of various means (i.e., sewn, heat fused) and hook or loop fastener 38 is attached to strap 35 by similar means. The end 36 of strap 35 is overcast-sewn or heat-cut in such a manner as to minimize edge roughness. Pad 39 is fabricated from a cushioning material and preferably coated with a non-porous, flexible material (e.g., vinyl).

To reduce transmission of communicable skin conditions, the nature of all materials and constructions used are such that the entire headgear may be quickly and easily disassembled for cleaning in the shower following practices or contests.

1. Protective headgear for wrestling and other activities, comprising:
a cap having an outer surface configured to cover the head of a wearer while leaving the face exposed;
a jaw strap for holding the cap in position while worn;
the cap including internally accessible side pockets associated with the ears of a wearer;
a pair of right and left ear guard inserts, each configured to be removably received by a respective one of the side pockets; and
wherein each ear protection insert is retained within its respective side pocket when the cap is worn and held in position with the jaw strap.

2. The protective headgear of claim 1, wherein each ear guard insert is slightly smaller in size than the side pockets such that each insert floats in its respective side pocket while worn.

3. The protective headgear of claim 1, including one or more flaps of material that at least partially cover each side pocket on the inside of the cap, thereby providing an entrance through which each ear guard insert is inserted and removed.

4. The protective headgear of claim 3, including two overlapping flaps of material disposed over each side pocket.

5. The protective headgear of claim 4, wherein the two overlapping flaps of material disposed over each side pocket provide substantially vertical entrances to receive and remove the respective ear guard inserts.

6. The protective headgear of claim 1, further including:
a face band and a neck band attached to the cap; and
wherein the face and neck bands are stiffer than the outer surface of the cap to transmit attachment loads.

7. The protective headgear of claim 1, wherein the cap is composed of a center panel and opposing side panels.

8. The protective headgear of claim 1, wherein the cap provides a continuous cover or wherein portions of the cap are omitted to allow for added ventilation.

9. The protective headgear of claim 1, wherein each ear guard insert is composed of a rigid or semi-rigid panel including a concave surface to receive a respective ear of a wearer.

10. The protective headgear of claim 9, further including one or more compressible spacer pads attached to the concave surface of each ear guard insert with one or more gaps between the compressible spacer pads to facilitate air flow around the ears of a wearer.

11. The protective headgear of claim 1, including at least a pair of apertures through the cap, at least one associated with each of the side pockets for to permit the transmission of air, water vapor or sound.

12. The protective headgear of claim 1, wherein the cap is constructed of a breathable material.
